(19) United States
(12) Patent Application Publication
(10) Pub. No.: US 2021/0145622 A1
(43) Pub. Date: May 20, 2021

(54) SELF-ADJUSTABLE SOLES

(71) Applicant: Hewlett-Packard Development Company, L.P., Spring, TX (US)

(72) Inventors: Felipe Riffel, Porto Alegre (BR); Josue Pereira, Porto Alegre (BR); Bruno Guedes, Porto Alegre (BR)

(73) Assignee: Hewlett-Packard Development Company, L.P., Spring, TX (US)

(21) Appl. No.: 16/629,625

(22) PCT Filed: Jul. 17, 2017
(86) PCT No.: PCT/US2017/042387

§ 371 Date: Jan. 9, 2020

(51) Int. Cl. A61F 5/14; A61F 5/32; A61F 5/34; A43B 13/20; A43B 3/00; A43B 7/14; A43B 13/18; A43B 17/00; A43B 17/02; A43B 17/03; B33Y 80/00; G01P 15/00; G01P 13/00

(52) U.S. Cl. CPC A61F 5/14; A61B 5/1038; A61F 5/34; A43B 13/203; A43B 3/0005; A43B 7/142; A43B 7/144; A43B 13/186; A43B 17/006; A43B 17/026; A43B 17/035; B33Y 80/00; G01P 15/00; G01P 13/00; A61F 5/32

(57) ABSTRACT

In example implementations, a self-adjustable sole is provided. The self-adjustable sole includes a soft sole, a flexible sole and a hard sole. The soft sole includes a first sensor. The flexible sole includes a bag containing an amount of fluid. The hard sole includes a controller that is in communication with the first sensor and the bag. The controller controls the amount of the fluid in the bag based on data collected by the sensor.

### BACKGROUND

Certain people may have back issues, or other medical issues, caused by foot issues, improper footwear, the way a person walks, and the like. Consumers may buy orthotic shoes to correct foot issues that may cause certain medical issues described above. Each consumer may have a unique foot problem. As a result, the orthotic shoes are customized to the foot of each individual.

Over time, a person's posture can change or the tread on a sole can wear down causing the person's posture to change. Changes to the person's posture, whatever the cause may be, may cause the consumer to purchase new customized orthotic shoes.

### BRIEF DESCRIPTION OF THE DRAWINGS

FIG. 1 is a block diagram of an exploded isometric side view of an example self-adjustable sole of the present disclosure;

FIG. 2 is a block diagram of an example flexible sole the present disclosure;

FIG. 3 is a block diagram of another example flexible sole of the present disclosure;

FIG. 4 is a block diagram of an example hard sole of the present disclosure;

FIG. 5 is a block diagram of an example charging station of the present disclosure; and

FIG. 6 is a block diagram of an example method for adjusting a sole of the present disclosure.

### DETAILED DESCRIPTION

The present disclosure relates to shoes that have self-adjustable soles. As discussed above, some consumers may use orthotic shoes to correct foot issues that may lead to other medical issues, such as back pain. Over time, a person's posture may change or the tread of the orthotic shoe may change. As a result, the person may periodically purchase new custom orthotic shoes. Getting fitted for the custom orthotic shoes can be time consuming and, depending on how often adjustments are made, the cost of the custom orthotic shoes can become quite expensive for the consumer.

Self-adjustable soles of the examples described herein may automatically adjust the sole based on user walking data that is collected over time. Thus, adjustments can be automatically made without having to purchase new custom orthotic shoes. This may allow the shoes to be worn over a longer period of time than custom orthotic shoes.

In addition, the shoes having the self-adjustable soles may be used by more than one person as the soles may be adjusted to the wearer. For example, sole profiles may be associated with different individuals and the soles may be adjusted based on the sole profile of the individual currently wearing the shoes.

Lastly, the examples described herein use multiple independent layers to form the self-adjustable soles. The multiple layers may allow the self-adjustable soles to feel like the inserts of “normal” shoes and allow individual layers to be replaced if a component within a particular layer fails rather than replacing the entire self-adjustable sole.

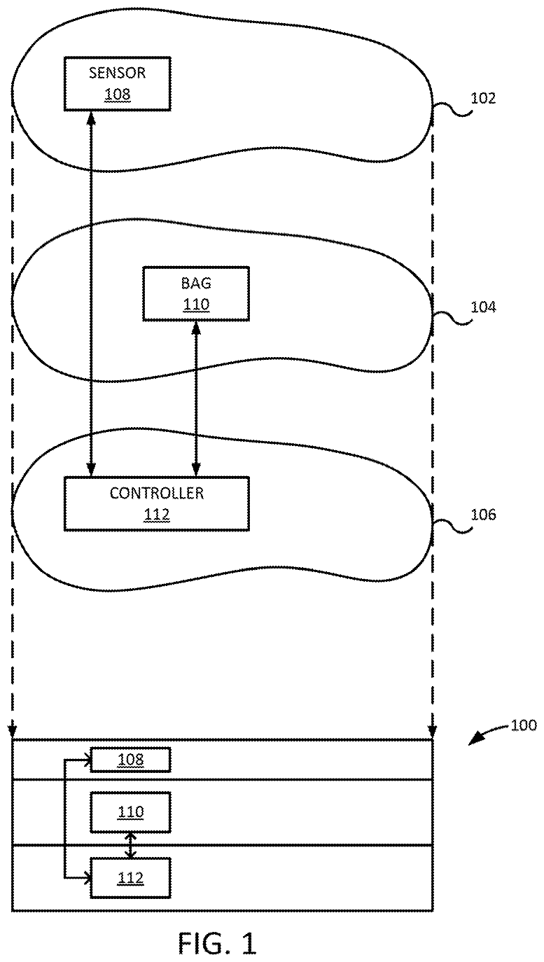

FIG. 1 illustrates a block diagram of an example self-adjustable sole 100 of the present disclosure. In one example, the self-adjustable sole 100 includes a soft sole 102, a flexible sole 104 and a hard sole 106. The soft sole 102, the flexible sole 104 and the hard sole 106 may be different independent layers that may be coupled together to form the self-adjustable sole 100.

In one example, the soft sole 102, the flexible sole 104, and the hard sole 106 may be fabricated via a three-dimensional (3D), or additive, printing process. For example, materials used for the soft sole 102, the flexible sole 104, and the hard sole 106 that are compatible with 3D printing may be used to fabricate each respective sole. The 3D printing process may be used to create the shell, housing, or enclosure of the soft sole 102, the flexible sole 104, and the hard sole 106. The respective components, discussed in further detail below, of the soft sole 102, the flexible sole 104, and the hard sole 106 may then be inserted into the shell, housing, or enclosure.

In one example, the soft sole 102 may include a sensor 108. The sensor 108 may be a pressure sensor. The sensor 108 may comprise a single sensor, or a plurality of sensors that are located throughout the soft sole 102. When a plurality of sensors 108 are deployed, the sensors 108 may be located in areas of the soft sole 102 that may collect pressure data related to how a user's foot presses against the soft sole 102 when walking or running.

In one example, the soft sole 102 may be formed from any material that is used to form a traditional sole. For example, the soft sole 102 may comprise a thermoplastic, a polypropylene, a foam, a gel, leather, a subortholen material, an acrylic, a composite carbon fiber, and the like.

The sensor 108 may be located within the soft sole 102. In other words, the sensor 108 is not located on a top surface of the soft sole 102 that would contact a user's foot directly. As a result, the soft sole 102 may be designed such that the self-adjustable sole 100 feels like a traditional insole for shoes. In other words, a user may not feel breaks, lines, bumps, grooves, unevenness caused by cut-outs, and the like, in the soft sole 102 due to components carved into the soft sole 102.

In one example, the flexible sole 104 may contain a bag 110 that contains an amount of fluid. The fluid may be any type of fluid such as air, water, oil, and the like. The flexible sole 104 may be located below, or underneath, the soft sole 102. The flexible sole 104 may be formed from any type of plastic or rubber material that can house the bag 110.

In one example, the bag 110 may also be referred to as a bladder or reservoir. The bag 110 may comprise any type of plastic material that can contain the fluid and resist tearing, popping, puncturing, and the like, under pressure applied by a user's foot when the user is walking or running.

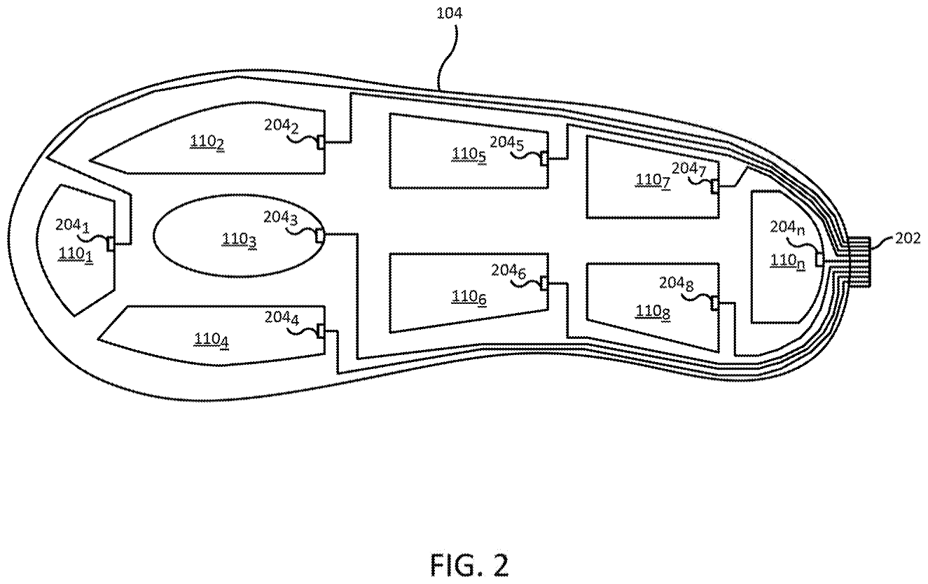

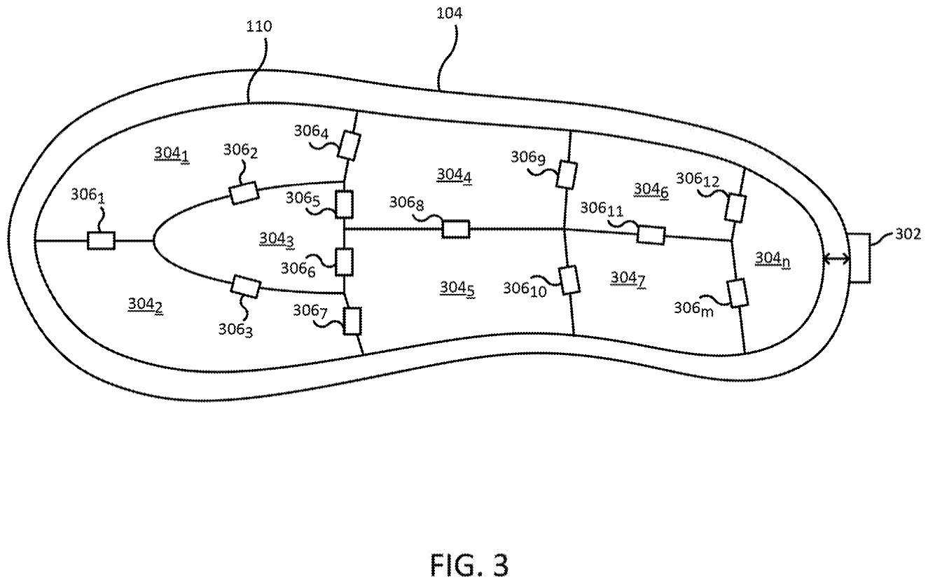

The amount of fluid in the bag 110 may be adjusted to provide the proper amount of support to the desired areas of a user's foot. Different designs of the bag 110 are illustrated in FIGS. 2 and 3. FIG. 2 illustrates an example of the flexible sole 104 that uses a plurality of bags 110₁ to 110ₙ (also referred to individually as a bag 110 or collectively as bags 110). In one example, each one of the bags 110₁ to 110ₙ may have a respective valve 204₁ to 204ₙ (also referred to individually as a valve 204 or collectively as valves 204) that is communicatively coupled to an interface 202. The valves 204 may be communicatively coupled via a respective micro-fluidic line that can carry fluid to and from a respective bag 110₁ to 110ₙ.

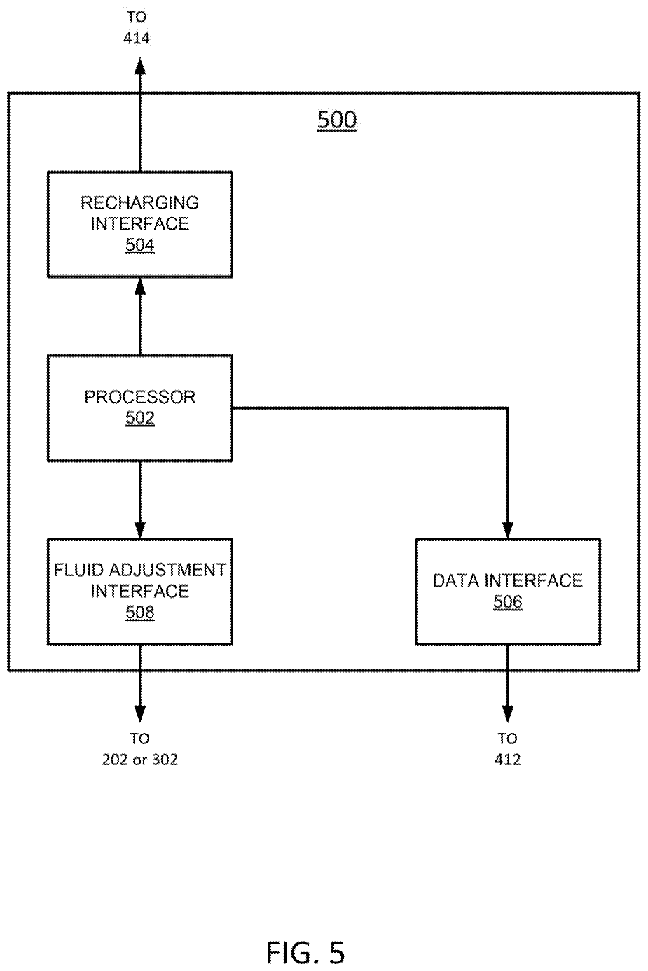

The interface may be coupled to a charging station 500, illustrated in FIG. 5 and described below. The interface may allow each bag 110₁ to 110ₙ to be independently filled with more or less fluid. As a result the amount of support provided by different bags 110₁ to 110ₙ in different areas of the flexible sole 104 may be independent controlled.

In one example, the bags may be arranged to cover substantially the entire surface area of the flexible sole 104. The number of bags 110 that are deployed may be a function of a desired level of customization. For example, the more bags 110 that are deployed in the flexible sole 104, the more granular level adjustments that can be made. For example, finer adjustments can be made to increase or decrease the amount of support provided to a particular area of a foot of a user.

In contrast, the less number of bags 110 that are deployed, the less granular level of adjustments that can be made. However, using less bags 110 may reduce overall costs and chances of failure.

In some examples, a large amount of smaller bags 110 may be deployed in certain areas to provide a high level of customization of the amount of support and other areas may use larger bags 110. For example, a higher level of granular control in the amount of support below a user's heel may be desired. Thus, a plurality of smaller bags 110 may be used in the heel area. However, less granular control may be desired along an outside of the foot, thus a single larger bag 110 may be used in the area along the outside of the foot.

FIG. 3 illustrates another example of the bag 110 in the flexible sole 104. The bag 110 may be a single bag that is approximately the same shape and size as the flexible sole 104. Different areas or compartments 304₁ to 304ₙ (also referred to herein individually as an area 304 or collectively as areas 304) may be created within the bag 110. For example, each area 304 may be created by creating a seal around each area 304 within the bag 110.

Access to each area 304 and transfer of fluid between areas 304 may be controlled by valves 306₁ to 306ₘ (also referred to herein individually as a valve 306 or collectively as valves 306). The valves 306 may be used to control the amount of fluid in each area 304. In one example, the valves 306 may be electro-mechanical valves that can be individually controlled via an interface 302 that may be coupled to the charging station 500 described below and illustrated in FIG. 5.

In one example, each area 304 may contain an initial amount of fluid. The fluid may be moved to different areas 304 by opening and closing select valves 306 to create pressure differentials that cause the fluid to move in a desired direction. In some examples, the charging station 500 described below and illustrated in FIG. 5, may inject additional fluid, or vacuum out fluid, to initiate the movement of the fluid from one area to another. The fluid may be moved to and from different areas 304 to provide more or less support in desired locations of a user's foot. For example, if the user desired more support in the arch (e.g., area 304₅) and less support in the heel (e.g., area 304ₙ), then fluid may be moved from the area 304ₙ to the area 304₅ via the area 304₇.

In one example, the hard sole 106 may be formed from a rigid plastic, wood, or other similar material. The material used to form the hard sole 106 may be rigid enough to protect components within the hard sole 106. For example, the hard sole 106 may include a controller 112 that is contained within the hard sole 106.

In one example, the controller 112 may be in communication with the sensor 108 and the bag 110. For example, the pressure data collected by the sensor 108 may be used by the controller 112 to control the amount of fluid in the bag 110. For example, the controller 112 may operate the valves 204 or 306 to control the amount of fluid in each bag 110 or area 304, respectively.

The sensor 108 may collect pressure data for different areas of the self-adjustable sole 100 over a period of time while the user is walking or running. Based on the pressure data, the controller 112 may cause an increase or a decrease in the amount of fluid in the bag 110, as discussed above, and in further detail below.

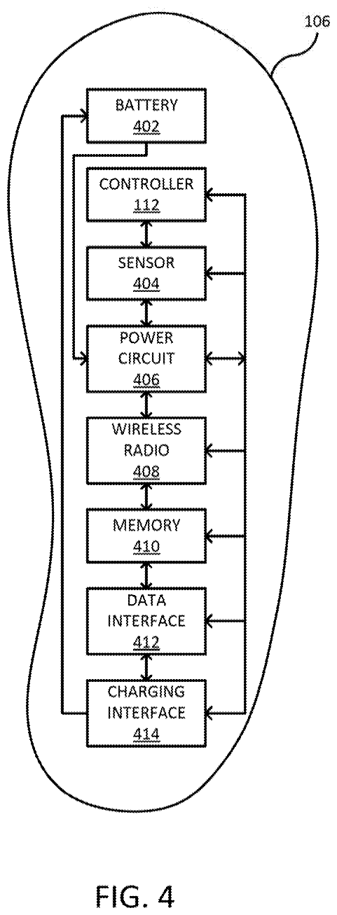

FIG. 4 illustrates another block diagram of the hard sole 106. In one example the hard sole 106 may include a battery 402, the controller 112, a second sensor 404, a power circuit 406, a wireless radio 408, a memory 410, a data interface 412, and a charging interface 414. The controller 112 may be in communication with, and control operation of, the sensor 404, the power circuit 406, the wireless radio 408, the memory 410, the data interface 412, and the charging interface 414.

The battery 402 may be coupled to the power circuit 406 to provide power to the controller 112 and the other components within the hard sole 106. The battery 402 may be a rechargeable battery such as a lithium ion battery, a nickel cadmium battery, and the like. The battery 402 may be coupled to the charging interface 414 and receive power to be recharged periodically by the charging station 500, as discussed below.

The power circuit 406 may provide electronic components, a bus, a circuit board, and the like, that electronically connect the components within the hard sole 106. The power circuit 406 may distribute power from the battery 402 to the other electronic components within the hard sole 106.

The second sensor 404 may be movement sensor. For example, the second sensor 404 may be an accelerometer or a gyroscope that can detect when the user is moving, walking, running, and the like. The controller 112 may detect when the user is walking based on the data collected by the second sensor 404. In response, the controller may send a control signal to the sensor 108 to begin collecting pressure data while the user is walking. Thus, in one example, data may be collected during movement of the user, rather than continuously even when the user is stationary, to conserve battery life of the battery 402.

The wireless radio 408 may transmit wireless signals to an endpoint device of the user. The endpoint device (not shown) may be any type of endpoint device such as a mobile telephone, smart phone, tablet computer, laptop computer, desktop computer, and the like. The wireless radio 408 may be a Bluetooth® radio, local area network (LAN) radio that transmit data via a Wi-Fi connection, and the like. The wireless radio 408 may allow the controller 112 to send graphical images, updates, data, and the like to the endpoint device of the user. For example, the graphical images may include an image of the bag 110 of the flexible sole 104 and the amounts of fluid in the bag 110 indicating how much support is provided at each location of the foot. The updates may include notifications when changes are made to the amount of fluid or when the amount of fluid is moved to different areas of the bag 110. The data may include battery life remaining, the amounts of steps the user has taken, a walking profile of the user based on the pressure data, and the like. For example, when the battery life remaining falls below a threshold (e.g., 10%, 20%, and the like) a notification may be transmitted to the endpoint device of the user.

The memory 410 may store the pressure data that is collected and any other data (e.g., number of steps taken, user profiles, and the like). In one example, different user profiles may be stored in the memory 410. For example, two different users with the same shoe size may share the shoes having the self-adjustable sole 100. Each user may have a different profile related to how the amount of fluid is distributed within the bag 110 of the flexible sole 104. For example, the user may communicate with the controller 112 with his or her endpoint device via the wireless radio 408 indicate which user profile should be activated. The controller 112 may then adjust the amount of fluid in the bags 110, or the different areas 304 of the bag 110, based on the selected user profile.

In another example, the user may have different user profiles for different movements. For example, the user may have a walking profile and a running profile. The user may prefer to have more support at the heel when walking and have more support in the balls of the foot when running. Thus, depending on whether the walking profile or the running profile is selected, the controller 112 may adjust the amount of fluid in the bags 110, or the different areas 304 of the bag 110, based on the selected movement profile.

The data interface 412 may connect to the charging station 500 to import or export data that is collected, the user profiles, and the like. For example, to keep the size and cost down for the self-adjustable sole 100, the processing capability of the controller 112 may be limited. The pressure data may be exported to a more powerful processor that can be used to generate the appropriate profiles used to determine the amount of fluid in each bag 110 or the amount of fluid in different areas 304 of the bag 110.

The charging interface 414 may be used to recharge the battery 402. For example, the charging interface 414 may be coupled to an external power source that can recharge the battery 402.

In one example, the soft sole 102, the flexible sole 104 and the hard sole 106 may each have a similar shape. The shape may be based on the shape of a shoe that the self-adjustable sole 100 will be inserted into. For example, the self-adjustable sole 100 may be designed to be coupled into a particular type of shoe. In another example, the shape may be a generic shape that allows the self-adjustable sole 100 to be inserted into a variety of different shoes.

As noted above, the self-adjustable sole 100 comprises three independent layers. The different independent layers allows the self-adjustable sole 100 to feel like a “regular” insole. For example, the soft sole 102 may be made with traditional insole materials and the sensors 108 may be inserted into the soft sole 102 rather than be cut into the soft sole 102 as with other designs.

In addition, the three independent layers allows each layer to be individually removed or repaired when components within a respective layer malfunction. In other words, a single layer may be replaced rather than replacing the entire self-adjustable sole 100, which may reduce overall costs and increase the life of the self-adjustable sole 100. For example, if the bag 110 malfunctions, then the flexible layer 104 may be replaced with a new flexible layer 104 rather than disposing of the entire self-adjustable sole 100 that may have a fully functioning sensor 108 in the soft sole 102 and fully functioning controller 112 in the hard sole 106.

FIG. 5 illustrates a block diagram of the charging station 500. The charging station 500 may include a processor 502, a recharging interface 504, a data interface 506, and a fluid adjustment interface 508. The charging station 500 may include additional components that are not shown such as a memory in communication with the processor 502, a power connection to a power outlet to provide power to the recharging interface 504, and the like.

Although it appears that the recharging interface 504, the data interface 506, and the fluid adjustment interface 508 are located in different ports, it should be noted that the recharging interface 504, the data interface 506, and the fluid adjustment interface 508 may be located in a single common port. In other words, a port in the self-adjustable sole 100 may include the interface 202 or 302, the data interface 412 and the charging interface 414. The single port may be coupled to a single port of the charging station 500 to connect the recharging interface 504 to the charging interface 414, the data interface 506 to the data interface 412 and the fluid adjustment interface 508 to the interface 202 or 302.

The processor 502 may be in communication with, and control operation of, the recharging interface 504, the data interface 506, and the fluid adjustment interface 508. In one example, the processor 502 may activate the recharging interface 504 when a connection to the charging interface 414 is detected.

The processor 502 may activate the data interface 506 when a connection to the data interface 412 is detected. The data interface 506 may be a wireless radio or a physical connection. For example, the data interface 506 may be a wireless radio that establishes a communication path with the wireless radio 408 to transfer data. In another example, the data interface 506 may be a physical connection (e.g., connection with physical pins, a universal serial bus (USB) connection, an Ethernet port/connection, and the like) that connects to the data interface 412.

When the connection to the data interface 412 is detected, the processor 502 may initiate transfer of any data stored in the memory 410 automatically to the memory in the charging station 500 (not shown). In another example, when the connection to the data interface 412 is detected, the processor 502 may signal the connection to the controller 112. The controller 112 may then send a notification to the endpoint device of the user via the wireless radio 408 and provide the user with a selectable graphical window that includes options to download data from the memory 410, or options to select specific data from the memory 410 that the user would like to download.

The processor 502 may activate the recharging interface 504 when a connection to the interface 202 or 302 is connected and based on the pressure data that is collected and/or a profile that is selected. For example, the user may periodically connect the self-adjustable sole 100 to the charging station 500 to recharge the battery 402. In response, the pressure data may be downloaded to the charging station 500, as described above, and analyzed by the processor 502.

For example, the processor 502 may determine that the user has gradually applied more pressure to an inner side of the heel. For example, as a user walks, the sole of the shoe may wear unevenly on the inside causing the user's foot to slide inward creating more pressure on the inner side of the heel. Thus, the processor 502 may determine that the support on the inner side of the heel may be increased by adding fluid to that area of the foot in the bag 110 of the flexible sole 104.

In another example, the pressure data may determine while the user is running that the user could benefit from more support in the arch of the user's foot and does not benefit from support in the heel of the user's foot. Thus, for a running profile of the user, the processor 502 may provide more support by adding fluid to arch area and removing fluid in the heel area of the bag 110 of the flexible sole 104.

Based on the analysis, the processor 502 may cause the fluid adjustment interface 508 to adjust the amount of fluid in one of the bags 110₁-110ₙ of the design illustrated in FIG. 2, or a particular area 304 in the bag 110 of the design illustrated in FIG. 3. The fluid adjustment interface 508 may include a reservoir of the fluid that allows fluid to be added via the fluid adjustment interface 508, or removed from the bag 110

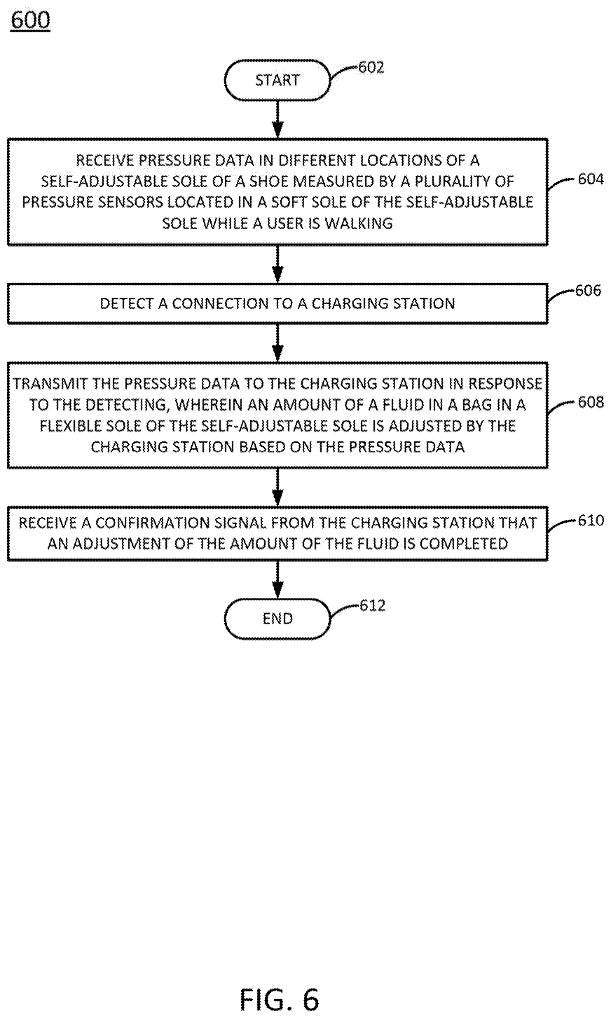

FIG. 6 illustrates a flow diagram of an example method 600 for adjusting a self-adjusting sole of the present disclosure. In one example, the method 600 may be performed by a controller 112 in the self-adjustable sole 100.

At block 602, the method 600 begins. At block 604, the method 600 receives pressure data in different locations of a self-adjustable sole of a shoe measured by a plurality of pressure sensors located in a soft sole of the self-adjustable sole while a user is walking. For example, an accelerometer or a gyroscope sensor in a hard sole of the self-adjustable sole may detect when a user begins walking. While the user is walking, the controller may signal the pressure sensors in a soft sole of the self-adjustable sole to begin collecting pressure data. For example, a different pressure sensor may be placed at different locations throughout the soft sole that correspond to a shape of the foot of the user. The pressure data that is collected may be for each pressure sensor in different locations. The pressure data may be used to generate pressure maps of the user's foot while the user is walking. The pressure map may indicate where additional support could be used to support the user's foot.

At block 606, the method 600 detects a connection to a charging station. At a later time, the user may connect the self-adjustable sole to the charging station to recharge the battery within the self-adjustable sole. For example, the battery may be a rechargeable battery that provides power for the electronic components (e.g., the sensors, the controller, and the like) within the self-adjustable sole to operate.

At block 608, the method 600 transmits the pressure data to the charging station in response to the detecting, wherein an amount of a fluid in a bag in a flexible sole of the self-adjustable sole is adjusted by the charging station based on the pressure data. After the self-adjustable sole is connected to the charging station, the pressure data may be transmitted to the charging station for analysis via a data interface connection. The processor of the charging station may perform the analysis (e.g., generating the pressure maps of the user's foot described above) and determine the particular locations or areas in the self-adjustable sole that can use more or less support.

In response, the charging station may adjust the amount of fluid in the bag via a fluid adjustment interface. For example, depending on the design of the bag that is deployed, the charging station may add or remove fluid to a particular bag of a plurality of bags, or move fluid around different areas of a single bag with separated areas or compartments. In one example, the processor of the charging station may control valves located on the bags or separating the different areas of the bag to control the movement of the fluid and the amount of fluid in the bag.

At block 610, the method 600 receives a confirmation signal from the charging station that an adjustment of the amount of the fluid is completed. For example, after the adjustments are completed, the processor of the charging station may send a confirmation signal to the controller. The controller may then transmit a confirmation that the adjustment is complete to an endpoint device of the user via a wireless radio in a hard sole of the self-adjustable sole. At block 612, the method 600 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A self-adjustable sole, comprising:
a soft sole, wherein the soft sole comprises a sensor;
a flexible sole, wherein the flexible sole comprises a bag containing an amount of a fluid; and
a hard sole, wherein the hard sole comprises a controller in communication with the sensor and the bag, wherein the controller controls the amount of the fluid in the bag based on data collected by the sensor.

2. The self-adjustable sole of claim 1, wherein the sensor comprises a plurality of pressure sensors located at various different locations on the soft sole.

3. The self-adjustable sole of claim 1, wherein the fluid comprises water, air, or oil.

4. The self-adjustable sole of claim 1, wherein the bag comprises a plurality of different bags located at various different locations within the flexible sole.

5. The self-adjustable sole of claim 4, wherein each one of the plurality of different bags comprises a respective valve to add the fluid or to remove the fluid.

6. The self-adjustable sole of claim 1, wherein the bag comprises a single enclosed bag comprising a plurality of different compartments having a respective control valve to control movement of the fluid between the plurality of different compartments

7. The self-adjustable sole of claim 1, the hard sole comprising:
a power circuit in communication with the controller;
a memory in communication with the power circuit and the controller;
a second sensor in communication with the power circuit and the controller;
a short range radio in communication with the power circuit and the controller;
a battery coupled to the power circuit; and
a charging interface coupled to the battery.

8. The self-adjustable sole of claim 7, wherein the second sensor comprises an accelerometer to track when a position of a foot of a user while the user is walking, wherein the data collected by the sensor is correlated to the position of the foot of the user that is tracked by the second sensor.

9. A charging station, comprising:
a fluid adjustment interface in communication with a bag containing an amount of a fluid in a flexible sole of a self-adjusting sole;
a recharging interface in communication with a charging interface in a hard sole of the self-adjusting sole to charge a battery of the self-adjusting sole;
a data interface in communication with a controller in the hard sole of the self-adjusting sole to collect sensor data stored in a memory of the self-adjusting sole; and
a processor in communication with the fluid reservoir interface, the recharging interface and the data interface, wherein the controller causes the fluid reservoir interface to adjust the amount of the fluid based on the sensor data.

10. The charging station of claim 9, wherein the bag comprises a plurality of different bags each having a respective control valve and the fluid reservoir interface comprises a connection to the respective control valve of each one of the plurality different bags.

11. The charging station of claim 10, wherein the fluid reservoir interface adjusts the amount of the fluid in the plurality of different bags by adding the fluid to one of the plurality of different bags when the sensor data associated with the one of the plurality of different bags is above a threshold.

12. The charging station of claim 9, wherein the data interface comprises a wireless radio or a physical connection.

13. A method, comprising:
receiving, by a controller, pressure data in different locations of a self-adjustable sole of a shoe measured by a plurality of pressure sensors located in a soft sole of the self-adjustable sole while a user is walking;
detecting, by the controller, a connection to a charging station;
transmitting, by the controller, the pressure data to the charging station in response to the detecting, wherein an amount of a fluid in a bag in a flexible sole of the self-adjustable sole is adjusted based on the pressure data by the charging station; and
receiving, by the controller, a confirmation signal from the charging station that an adjustment of the amount of the fluid is completed.

14. The method of claim 13, comprising:
transmitting, by the controller, a notification to an endpoint device of the user when a charging operation and the adjustment are completed.

15. The method of claim 13, comprising:
transmitting, by the controller, a notification to an endpoint device of the user when a battery of the self-adjustable sole is below a threshold.
